(19) United States
(12) Patent Application Publication
(10) Pub. No.: US 2007/0009445 A1
(43) Pub. Date: Jan. 11, 2007

(54) Aerosol compositions and methods

(75) Inventor: Charles R. Eck, Shrewsbury, MA (US)

Correspondence Address: SULLIVAN & WORCESTER LLP, ONE POST OFFICE SQUARE, BOSTON, MA, 02109, US

(73) Assignee: Xemplar Pharmaceuticals, LLC, Fall River, MA (US)

(21) Appl. No.: 11/477,158

(22) Filed: Jun. 28, 2006

Related U.S. Application Data
(60) Provisional application No. 60/697,717, filed on Jul. 8, 2005.

(51) Int. Cl. A61K 9/14; A61K 31/202; A61L 9/04

(52) U.S. Cl. 424/46; 514/560

(57) ABSTRACT

The present invention provides drug formulations and methods that comprise omega-3 and/or omega-6 fatty acids, and their ester derivatives (e.g., methyl, ethyl, isopropyl, etc.), which are soluble in non-CFC propellants. The addition of omega-3 or omega-6 fatty acids to the aerosol formulations also provides therapeutic benefits.

### CROSS-REFERENCE TO RELATED APPLICATION

This application claims priority to U.S. Provisional Patent Application No. 60/697,717, filed on Jul. 8, 2005.

### FIELD OF THE INVENTION

The invention relates to aerosol compositions comprising omega-3 and/or omega-6 fatty acids.

### BACKGROUND OF THE INVENTION

Drugs for treating respiratory or nasal disorders are frequently administered in aerosol formulations through the mouth or nose. One widely used method for dispensing such aerosol drug formulations involves making a suspension formulation of the drug as a finely divided powder in a liquefied gas, known as a propellant. The suspension is stored in a sealed container capable of withstanding the pressure required to maintain the propellant as a liquid. The suspension is usually delivered by activation of a dose metering valve affixed to the container. This system is commonly referred to as a pressurized metered dose inhaler (pMDI). Users of suspension pMDIs are always instructed to shake the container well before use. However, even a short interval between the conclusion of shaking and the act of dispersing a charge from the aerosol unit may be sufficient to allow some sedimentation of the suspension to occur. This possibility represents a particular problem where the suspended material is a medicine, since it can result in the patient receiving a dose, which, although of the correct volume, contains either too little or too much of the medicine. To prevent the rapid settling of the suspension, surfactants are commonly employed. The surfactants are generally soluble in the propellant and serve to coat the fine drug particles when they are mixed. The surfactant coated particles are less likely to be attracted to one another to form aggregates and agglomerates. In this “neutralized” state, the drug suspension formed is more stable and settles or creams at measurably slower rates. Such “stabilized” suspensions are desirable because dosing and delivery is more reproducible, and the suspensions have a longer shelf life.

Urgent measures introduced in the 1980's to protect the ozone layer resulted in a ban on the production and use of fully halogenated chlorofluorocarbon (CFC) propellants in the developed world. Only “essential” uses like medicinal therapeutics were exempt from the ban. As the pharmaceutical industry moved forward to find effective substitutes for CFCs and CFC formulations, they encountered the problem that none of the classically employed surfactants were suitable (e.g., they had negligible solubility) for use with non-CFC substitute propellants, such as HFA propellants 1,1,1,2 tetrafluoroethane, also known as HFA 134a, and 1,1,1,2,3,3,3,heptafluoropropane, also known as HFA 227. See U.S. Pat. No. 6,743,413, incorporated herein by reference.

Over the past ten or more years, the pharmaceutical industry has expended much effort in identifying appropriate surfactants. Despite the fact that some surfactants have been suggested and patented, for example those disclosed in European Patent No. 0327777, none of these surfactants have actually been used in any of the currently approved HFA products without the use of a co-solvent (i.e., ethanol). In fact, several of the approved suspension products do not contain any surfactant, but rather componentry “tricks” are employed to offset the poor suspension quality. The problem of rapid settling of drug particle suspensions is particularly acute in the development of non-CFC aerosol formulations using propellants HFA 134a and HFA 227.

A need therefore remains for a surfactant with improved solubility for use with non-CFC propellants.

### SUMMARY OF THE INVENTION

The present invention provides drug formulations and methods that comprise omega-3 fatty acids and/or omega-6 fatty acids and/or their ester derivatives (e.g., methyl, ethyl, isopropyl, etc.), which are soluble in non-CFC propellants such as, for example, HFA 134a and HFA 227. The addition of omega-3 and/or omega-6 fatty acids to the aerosol formulations also provides therapeutic (e.g., nutriceutical) benefits. The invention thus provides improved (e.g., more stable) aerosol suspension-based products over current formulations.

In one aspect, the invention provides non-CFC propellant drug particle formulations comprising omega-3 and/or omega-6 fatty acid-coated particles. In certain embodiments, the drug particle formulation is a fluid or aerosol suspension. The drug particle formulations comprise a plurality of drug particles, a propellant substantially free of CFCs, and a soluble surfactant comprising an omega-3 and/or an omega-6 fatty acid ester. The omega-3 and/or omega-6 fatty acid ester prevents or reduces the amount of aggregation, agglomeration, caking, and/or precipitation (e.g., crystallization).

In certain embodiments, the propellant comprises 1,1,1,2 tetrafluoroethane and/or 1,1,1,2,3,3,3 heptafluoropropane. In other embodiments, the propellant comprises a non-chlorofluorocarbon chemical such as a hydrocarbon, nitrogen, argon, nitrous oxide, air, and/or carbon dioxide. The hydrocarbon may be n-butane, isobutane, propane, pentane, isopentane, and/or isobutene.

In certain embodiments, the omega-3 or omega-6 fatty acid ester comprises a methyl ester, an ethyl ester, and/or an isopropyl ester. In another embodiment, the omega-3 or omega-6 fatty acid ester comprises a glycerol, sorbitol, or other alcohol ester.

Suitable omega-3 fatty acid esters include an ester of linoleic, linolenic, eicosapentaenoic, and docoashexaenoic acid, such as, for example, an isopropyl ester of omega-3 linoleic acid, an isopropyl ester of alpha linoleic acid, an isopropyl ester of eicosapentaenoic acid, and an isopropyl ester of docosahexaenoic acid.

Suitable omega-6 fatty acid esters include an isopropyl ester of linolenic acid and an isopropyl ester of gamma-linoleic acid.

In another aspect, the invention provides a delivery device comprising the drug formulations of the invention described herein. The delivery device may comprise a container, a valve, and an actuator. In an embodiment, the delivery device is a metered dose inhaler. The container may be made of coated or non-coated aluminum, steel, or glass, for example.

In another aspect, the invention provides a method for preparing a drug particle formulation comprising fine drug particles that are resistant to aggregation, agglomeration, caking, and/or precipitation. The method includes the steps of (a) combining (i) an omega-3 and/or omega-6 fatty acid, or ester thereof, (ii) a propellant substantially free of CFCs, and (iii) a plurality of fine drug particles to form a particle suspension; and (b) homogenizing the particle suspension. The homogenized particle suspension is resistant to aggregation, agglomeration, caking, and/or precipitation. In an embodiment, the homogenizing step comprises high shear mixing.

In an embodiment, the omega-3 and/or omega-6 fatty acid, or ester thereof, is combined with the propellant prior to being combined with the plurality of fine drug particles. In another embodiment, the drug particles are pre-coated with an omega-3 and/or omega-6 fatty acid, or ester thereof, prior to being combined with the propellant. In another embodiment, the omega-3 and/or omega-6 fatty acid, or ester thereof, is dissolved in the propellant prior to being combined with the drug particles. The method may comprise the additional step of filtering or precipitating and/or isolating the coated particles from the suspension.

In another aspect, the invention provides a method for treating a respiratory, nasal, or systemic disorder. The method includes the steps of preparing a fine particle suspension according to the methods of the invention and administering the suspension to a patient to a mucous membrane, for example, of an oral, pulmonary, buccal or nasal passage.

In another aspect, the invention provides methods for preparing a coated drug particle coated with an omega-3 and/or omega-6 fatty acid. The method comprises the steps of (a) providing to a first vessel an omega-3 and/or omega-6 fatty acid; (b) providing to the vessel a propellant substantially free of CFCs; (c) providing to the vessel a plurality of fine drug particles to form a particle suspension; (d) homogenizing the particle suspension; (e) spraying the homogenized particle suspension onto a surface, thereby forming micron sized droplets comprising propellant and omega-3 and/or omega-6 fatty acid coated drug particles; and (f) isolating the coated drug particles. In an embodiment, the homogenized particle suspension is sprayed onto the interior wall of a second vessel via a spray nozzle that is located either on the first vessel or the second vessel. In an embodiment, the atmospheric pressure of the second vessel is about 0.001 to about 1 atmosphere, for example about 1 atmosphere. The second vessel may be warmed to accelerate the evaporation of fluid and is kept at a temperature between about 10° C. to about 100° C., about 20° C. to about 40° C., or about 30° C. to about 40° C., for example.

In a particular embodiment, the first vessel comprises a formulation tank and the second vessel comprises a dispensing vessel in fluid communication with the formulation tank. The formulation tank and the dispensing vessel are connected via a transfer line such that the fine drug particles are added to the dispensing vessel and are flushed into the formulation tank with a propellant via the transfer line. In an embodiment, the formulation tank is kept under constant stirring conditions, for example about 500 rpm. The method may further comprise the step of flushing the contents of the transfer line back into the formulation tank using nitrogen after the homogenizing step.

In an embodiment, the isolating step comprises dessicating (e.g., evaporating) the formulation, wherein the drug particles are deposited on the surface. In a particular embodiment, the isolating step comprises collecting the drug particles from the interior walls of the second vessel. In another embodiment, the method comprises the step of attaching the second vessel to a final formulation vessel and the coated particles are flushed from the second vessel into the final formulation vessel with a fluid. The fluid may be a non-CFC propellant, such as for example, HFA 134a and HFA 227, or a combination thereof, or may be a CFC propellant. Although the methods of the invention may be performed in any of a number of different orders, the steps of the above method can be performed in the order of (a)-(b)-(c)-(d)-(e)-(f) or (b)-(a)-(c)-(d)-(e)-(f), for example.

### BRIEF DESCRIPTION OF THE DRAWINGS

The foregoing and other objects, features and advantages of the present invention, as well as the invention itself, will be more fully understood from the following description of preferred embodiments when read together with the accompanying drawing, in which:

FIG. 1 provides an illustration of the vessels used in the preparation of a drug particle coated with omega-3 and/or omega-6 fatty acids.

### DETAILED DESCRIPTION OF THE INVENTION

The present invention provides drug formulations and methods that comprise omega-3 fatty acids and/or omega-6 fatty acids and/or their ester derivatives (e.g., methyl, ethyl, isopropyl, etc.), which are soluble in non-CFC propellants.

Omega-3 fatty acids are considered to be essential fatty acids, meaning they are essential to human health but cannot be manufactured by the body. Therefore, they must be obtained from food, such as fish and certain plant oils. Also known as polyunsaturated fatty acids (PUFAs), they play a crucial role in brain function and normal growth and development. There are three major types of omega fatty acids that are ingested in foods and used in the body, alpha linoleic acid (ALA), eicosapentaenoic acid (EPA), and docosahexaenoic acid (DHA). These materials have been found to lower total cholesterol and triglyceride levels in people with high cholesterol, lower blood pressure in people with hypertension, generally lower the risk of heart disease, help protect people against stroke, can help lower triglycerides and raise HDL in diabetics, reduce tenderness in joints and morning stiffness in people with rheumatoid arthritis, help increase levels of calcium in the blood, deposit calcium in the bones of people suffering from osteoporosis, and reduce feelings of depression and mania in people suffering from depression, bipolar disorder, and schizophrenia, for example. Some research suggests that the omega-3 fatty acids may decrease inflammation and improve lung function in adults with asthma.

Suitable non-CFC propellants include halogenated alkanes, such as the hydrofluoroalkanes HFA 134a and HFA 227, HFA 125, HFA 141b, HFA 152a, HFA 225, FC-C51-12 (perfluorodimethylcyclobutane), and DYMEL A (dimethyl ether). However, both CFC and non-CFC aerosol propellants may be used with the compositions and methods of the present invention. Useful CFC propellants include, for example, the traditionally used chlorofluorocarbons (i.e., Propellant 11 (trichlorofluoromethane), Propellant 12 (dichlorodifluoromethane) and Propellant 114 (dichlorotetrafluoroethane)).

It is expected that the compositions and methods of the invention will be suitable for the administration of a wide variety of peptide and non-peptide drugs. Examples of peptides that may be suitable are interferons and other macrophage activation factors, such as lymphokines, muramyl dipeptide (MDP), gamma-interferon, interleukins, and interferons alpha and beta, and related antiviral and tumoricidal agents; opioid peptides and neuropeptides, such as enkaphalins, endorphins and dynorphins, and related analgesics; renin inhibitors including new-generation anti-hypertensive agents; cholecystokinins (CCK analogs) such as CCK, ceruletide and eledoisin, and related cardiovascular- and CNS-targeting agents; leukotrienes and prostaglandins, such as oxytocin, and related antiinflammatory, oxytocic and abortifacient compounds; erythropoietin and analogs thereof, as well as related haematinics; LHRH and somatostatin analogs, such as leuprolide, buserelin, nafarelin and octreotide, and related down-regulators of pituitary receptors; parathyroid hormone and other growth hormone analogs; enzymes, such as DNase, catalase and alpha-1 antitrypsin; immunosuppressants such as cyclosporin; GM-CSF and other immunomodulators; and insulin. Such peptides or peptide analogs are frequently not well-absorbed when given orally.

Non-peptides that may readily be delivered using the formulation compositions and methods of the present invention include virtually any drug or medicament for which a pulmonary, nasal, other mucosal route, or systemic delivery route is deemed suitable. Generally categories include bronchodilators, including beta-agonists, such as isoproterenol, albuterol, isoetherine, metaproterenol, formoterol, and salmeterol and related anti-asthmatics; hormones; anti-inflammatory steroids, such as flunisolide, fluticasone, mometasone, budesonide, beclomethasone, triamcinolone, and similar anti-asthmatics; sulfonamides, such as, sulfamethoxazole for example, a vasoconstrictive amine such as brimonidine tartrate; an enzyme recombinant human deoxyribonuclease I (rhDNase); an alkaloid such as cocaine; cholinergic agents, such as ipratropium bromide, tiotropium, cromolyn, and related anti-asthmatics; and 5-lipoxygenase inhibitors (i.e., physiologically active compounds capable of affecting leukotriene biosynthesis, including leukotriene antagonists) and related leukotriene inhibitors. Examples of 5-lipoxygenase inhibitors include zileuton. Such bronchodilator medicaments may lend themselves to oral administration, but when given by inhalation are found to produce rapid reversal of bronchoconstriction in cases of allergic airway disease and asthma. Also, these compounds may be administered more frequently and at lower doses as pMDI formulations than when administered orally.

Suitable drugs include those that are adaptable for inhalation administration, for example, anti-allergic, respiratory (e.g., anti-asthmatic and bronchodilating), anti-histamines, anti-tussives, antibiotic, antiinflammatory, antifungal, analgesic, antiviral, anti-anxiety, sleep aids, anti-migraine, and cardiovascular drugs, and anginal preparations. Especially useful drugs include the respiratory drugs albuterol, salmeterol and amiloride, fluticasone esters, beclomethasone esters and (−)-4-amino-3,5-dichloro-α-[[[6-(2-pyridinyl)ethoxy]hexyl]amino]methyl] benzenemethanol. The invention also contemplates, combinations of drugs, in particular, synergistic combinations of drugs such as fluticasone and salmeterol.

Other suitable drugs include Isoproterenol [alpha-(isopropylaminomethyl) protocatechuyl alcohol], phenylephrine, phenylpropanolamine, glucagon, adrenochrome, trypsin, epinephrine, ephedrine, nicotine, codeine, atropine, heparin, morphine, dihydromorphinone, ergotamine, dehydroergotamine, scopolamine, methapyrilene, cyanocobalamin, terbutaline, rimiterol, flunisolide, colchicine, pirbuterol, orciprenaline, fentanyl, and diamorphine. Suitable antibiotics include neomycin, streptomycin, penicillin, procaine penicillin, tetracycline, tobramycin, chlorotetracycline and hydroxytetracycline; adrenocorticotropic hormone and adrenocortical hormones, such as cortisone, hydrocortisone, hydrocortisone acetate and prednisolone; insulin, anti-allergy compounds such as cromolyn sodium, etc. are also suitable.

U.S. Pat. No. 3,644,353, incorporated herein by reference, teaches a group of bronchodilating compounds that are particularly useful in the treatment of asthma and other respiratory diseases. The preferred compound taught therein is α¹-tert-butylaminomethyl-4-hydroxy-m-xylene α¹,α³-diol, also known in the United States by its generic name, “albuterol” and, in most other countries, “salbutamol.” This compound, especially in aerosol form, has been widely accepted by the medical community in the treatment of asthma.

Salmeterol, chemically named 4-hydroxy-α′-[[[6[(4-phenylbutyl)oxy]hexyl]amino] methyl]-1,3-benzene dimethanol, disclosed in British Patent Application No. 8,310,477, is a second generation bronchodilator that is longer acting.

The genetic disease cystic fibrosis is characterized by abnormalities that result in excessive pulmonary secretion, which can make breathing difficult. U.S. Pat. No. 4,501,729, incorporated herein by reference, discloses the use of the drug amiloride in an aerosol formulation to reduce the excess secretion.

United Kingdom Patent Specification No. 2088877 discloses fluticasone esters. Fluticasone esters are corticosteriods having topical anti-inflammatory action. Corticosteroids may be used in the management of patients whose asthma is inadequately treated by bronchodilators and/or sodium cromoglycate.

A further class of corticosteroids having topical anti-inflammatory action, beclomethasone esters, are described in United Kingdom Patent Specification No. 1 047 519. (−)-4-Amino-3,5-dichloro-α-[[[6-[2-(2-pyridinyl)ethoxy]hexyl]amino]methyl]benzenemethanol is a bronchodilator.

Drugs useful in the compositions and methods of the present invention include not only those specifically named above, but also where appropriate the pharmaceutically acceptable salts, esters, amides and prodrugs thereof. “Pharmaceutically acceptable salts, esters, amides and prodrugs” include those carboxylate salts, amino acid addition salts, esters, amides, and prodrugs of a compound that are, within the scope of sound medical judgment, suitable for use in contact with the tissues of humans and animals without undue toxicity, irritation, allergic response or the like, commensurate with a reasonable benefit/risk ratio and effective for their intended use.

In particular, the term “salts” refers to the relatively non-toxic, inorganic and organic acid addition salts of a medicinal compound. These salts can be prepared in situ during the final isolation and purification of the compound or by separately reacting the purified compound in its free base form with a suitable organic or inorganic acid and isolating the salt thus formed. Representative salts include the hydrobromide, hydrochloride, sulfate, bisulfate, phosphate, nitrate, acetate, oxalate, valerate, oleate, palmitate, stearate, laurate, borate, benzoate, lactate, phosphate, tosylate, citrate, maleate, fumarate, succinate, tartrate, naphthylate, mesylate, glucoheptonate, lactiobionate and laurylsulphonate salts and the like. These may include cations based on the alkali and alkaline earth metals, such as sodium, lithium, potassium, calcium, magnesium and the like, as well as nontoxic ammonium, quaternary ammonium and amine cations known in the art to be pharmaceutically acceptable, including, but not limited to, glycine, ethylene diamine, choline, diethanolamine, triethanolamine, octadecylamine, diethylamine, triethylamine, 1-amino-2-propanol-amino-2-(hydroxymethyl)propane-1,3-diol and 1-(3,4-dihydroxyphenyl)-2 isopropylaminoethanol, tetramethylammonium, tetraethylammonium, methylamine, dimethylamine, trimethylamine, ethylamine and the like. (See, for example, S. M. Berge, et al., “Pharmaceutical Salts,” J. Pharm. Sci., 66:1-19 (1977), incorporated herein by reference.) For use in the invention, albuterol will preferably be in the form of the sulphate salt or the free base and salmeterol will preferably be in the form of its 1-hydroxy-2-naphthoate salt. A suitable fluticasone ester for use in the invention is fluticasone propionate, and a suitable beclomethasone ester is beclomethasone dipropionate.

The following salts of the drugs mentioned above may be used: acetate, benzenesulphonate, benzoate, bicarbonate, bitartrate, bromide, calcium edetate, camsylate, carbonate, chloride, citrate, dihydrochloride, edetate, edisylate, estolate, esylate, fumarate, fluceptate, gluconate, glutamate, glycollylarsanilate, hexylresorcinate, hydrobromide, hydrochloride, hydroxynaphthoate, iodide, isethionate, lactate, lactobionate, malate, maleate, mandelate, mesylate, methylbromide, methylnitrate, methylsulphate, mucate, napsylate, nitrate, pamoate (embonate), pantothenate, phosphatediphosphate, polygalacturonate, salicylate, stearate, subacetate, succinate, sulphate, tannate, tartrate, and triethiodide.

Examples of pharmaceutically acceptable, non-toxic esters of a compound include (C₁-to-C₆ alkyl) esters wherein the alkyl group is a straight or branched chain. Acceptable esters also include (C₅-to-C₇ cycloalkyl) esters as well as arylalkyl esters such as, but not limited to, benzyl; (C₁-to-C₄ alkyl) esters are preferred.

Examples of pharmaceutically acceptable, non-toxic amides of medicinal compounds include amides derived from ammonia, primary (C₁-to-C₆ alkyl) amines and secondary (C₁-to-C₆ dialkyl) amines wherein the alkyl groups are straight or branched chain. In the case of secondary amines the amine may also be in the form of a 5- or 6-membered heterocycle containing one nitrogen atom. Amides derived from ammonia, (C₁-to-C₃ alkyl) primary amides and (C₁-to-C₂ dialkyl) secondary amides are also suitable. Amides of the compounds of the invention may be prepared according to conventional methods.

In addition to omega-3 and omega-6 fatty acids it may be desirable to add other excipients to an aerosol formulation to improve drug delivery, shelf life and patient acceptance. Such optional excipients include, but are not limited to, coloring agents, taste masking agents, buffers, antioxidants and chemical stabilizers.

Inhalation drugs, or a pharmaceutically acceptable salt hereof, are made particulate, e.g., by micronization, spray drying, supercritical fluid technologies, etc. by, for example, conventional jet mill micronization to no greater than 100 microns diameter, since larger particles may clog the valve or orifice of the container. Preferably, the particle size should be less than 25 microns in diameter. The particle size of the finely-divided solid powder should for physiological reasons be less than 25 microns and preferably less than about 10 microns in diameter. The particle size of the powder for inhalation therapy should preferably be in the range 2 to 10 microns. There is no lower limit on particle size except that imposed by the use to which the aerosol produced is to be put. Where the powder is a solid medicament, the lower limit of particle size is that which will be readily absorbed and retained on or in body tissues. When particles of less than about one-half micron in diameter are administered by inhalation they tend to be exhaled by the patient.

The production of the aerosol formulations of the invention using omega-3 and/or omega-6 fatty acids and their esters as surfactants utilizes classical aerosol manufacturing techniques. For example, the surfactant is weighed or otherwise measured out into an appropriate transfer container and either added directly to a batching vessel or to an addition port on a pressure batching vessel. Once the requisite amount of propellant is placed into the vessel (according to the batch record), the surfactant is introduced (e.g., by flushing with the necessary amount of propellant) and, optionally, other excipients are added through the addition port and into the pressure vessel where it is stirred for a sufficient time to allow solubilization. Next, the propellant/surfactant/drug suspension is homogenized for a period of time to form the homogenized suspension formulation. Once the suspension is made, the product is pressure filled through the valve into a product canister capable of withstanding the vapor pressure of the propellant and pre-fitted with a metering valve. Prior to use, the completed pMDI is shaken vigorously to form a homogeneous suspension.

Alternatively, a pMDI can also be produced by adding drug, surfactant and liquefied propellant (chilled below it's boiling point) to the batching vessel, stirred and homogenized for appropriate periods and then accurately transferred into the canister and a metering valve fitted to the container. This process is generally referred to as a “cold filling” process. The completed pMDI can then be brought to ambient temperature and prior to use, shaken vigorously to reform the homogeneous suspension prior to use.

The compositions of the invention may be prepared by combining the omega-3 and/or omega-6 fatty acid with a medicament that has been milled or otherwise reduced to a desired particle size, and placing the mixture in a suitable aerosol batching or pressure vessel. After mixing and homogenizing, the product is pressure filled into the pre-sealed container. Alternatively, the omega-3 or omega-6 fatty acid and medicament may be milled together after addition of propellant. In some instances, it may be necessary to wet-mill the medicament in a closed system, as for example under temperature and pressure conditions that permit the medicament to be milled while mixed with a liquid-phase aerosol propellant. It is expected that, for any particular combination of medicament, propellant and omega-3 and/or omega-6 fatty acid, the ideal order of addition of ingredients and the conditions under which they are combined may readily be determined.

In addition to suspension formulations, the compositions of the invention may be prepared as solutions. Similar to the suspension formulations, solution formulations may be prepared by combining the omega-3 and/or omega-6 fatty acid, a medicament and a propellant in any manner described above, but without the step of milling or the requirement for milled medicament. Thus the only requirement is to blend the components, whether they are combined in a single step (i.e., altogether) or in multiple steps. When the components are combined in multiple steps, the solution should be agitated after each addition to thoroughly blend and solubilize the components.

The invention also provides methods for preparing omega-3 and/or omega-6 fatty acid-coated particles, as well as methods for preparing and isolating the coated particles. For example, omega-3 linoleic acid isopropyl ester is solubilized in HFA 134a or HFA 227, a fine particle medicament is added and the suspension is homogenized and coated particles are isolated by filtration or spray drying. The resulting medicament particles are surfaced coated, for example, with a layer of omega-3 linoleic acid, isopropyl ester. These particles have unique physical properties such as improved formulation, stability, product absorption, bioavailability, and other synergistic therapeutic effects.

pMDIs prepared according to the teachings herein may be used in the same way as currently marketed pMDIs that use CFCs or hydrocarbon propellants. For example, in the case of albuterol, the amount of drug, surfactant and propellant can be adjusted to deliver 90 μg per valve actuation, the dose delivered in the currently marketed albuterol pMDIs.

When used in the above compositions, a therapeutically effective amount of a medicament of the present invention may be employed in pure form or, where such forms exist, in pharmaceutically acceptable salt, ester or prodrug form. By a “therapeutically effective amount” of a medicament is meant a sufficient amount of the compound to obtain the intended therapeutic benefit, at a reasonable benefit/risk ratio applicable to any medical treatment. It will be understood, however, that the total daily usage of the medicaments and compositions of the present invention will be decided by the attending physician within the scope of sound medical judgment. The specific therapeutically effective dose level for any particular patient and medicament will depend upon a variety of factors including the disorder being treated and the severity of the disorder; activity of the specific compound employed; the specific composition employed; the age, body weight, general health, sex and diet of the patient; the time of administration, route of administration, and rate of excretion of the specific medicament employed; the duration of the treatment; drugs used in combination or coincidental with the specific compound employed; and like factors well known in the medical arts. For example, it is well within the skill of the art to start doses at levels lower than required to achieve the desired therapeutic effect and to gradually increase the dosage until the desired effect is achieved.

The total daily doses of the medicaments contemplated for use with this invention, and consequently the concentrations by weight of the medicaments in the respective compositions, may vary widely, but are within the typical skill of the routine practitioner. In the case of an LHRH analog, such as leuprolide acetate, the intended daily dose may range from about 0.01 to about 5 mg/day; accordingly, where an aerosol inhaler is to be used several times a day with a discharge volume of between about 25 and about 150 μL, the concentration of medicament will be between about 0.05 and about 2.5 mg/spray. Similarly, in the case of a 5-lipoxygenase inhibitor expected to be administered in a daily dose ranging from about 0.1 to about 10 mg/kg/day, the concentration will be between about 0.1 and about 100 mg/mL. Of course, medicament concentrations outside of these ranges may also be suitable, where different potencies, dosing frequencies and discharge volumes are used.

In an embodiment of the drug formulation of the invention, the omega-3 and/or omega-6 fatty acid ester comprises about 0.001% to about 10%, about 0.01% to about 1%, about 0.01% to about 0.1%, or about 0.01% of the total weight of the drug formulation. The ratio of surfactant to drug is from about 1:100 to about 1:0.5 by weight, preferably in the range of about 1:50 to about 1:1 and most preferably in the range of about 1:25 to about 1:1 by weight. The amount of propellant can be varied according to the amount of drug formulation to be delivered with each activation of the metering valve. Typically, for an inhalation drug, the amount of propellant for each formulation of active drug depends on the volume of the metering valve and the dose desired. However the ratio of active drug or drugs to propellant is in the range from about 1:100 to about 1:4000 by weight. For example, for albuterol in an aerosol inhalation system outfitted with a Bespak BK300 valve, 18 g of propellant is utilized per 20 mg of albuterol to deliver an effective dose of albuterol.

The vapor pressure of a propellant system is an important factor as it provides the propulsive force for the medicament. The vapor pressure of the formulations at 25° C. is generally in the range about 20 to about 150 psig (1.4 to 1.3×10⁵ N/m²) preferably in the range about 40 to about 90 psig (2.8 to 6.2×10⁵ N/m²), for example, about 60 psig.

Practice of the invention will be still more fully understood from the following examples, which are presented herein for illustration only and should not be construed as limiting the invention in any way.

### EXEMPLIFICATION

### Example 1

### Preparation of an Aerosol Formulation Containing Fluticasone Propionate

The formulations reported in the following table were prepared. The examples in Table 1 represent different proportions of fluticasone propionate (FP) to isopropyl linoleate, for example that represent different commercial doses, or amounts of the drug per formulation. For example, Example 1 represents a 50 μg drug/dose formulation, examples 2 and 4 represent a 125 μg drug/dose formulation, and examples 3 and 5 represent a 250 μg drug/dose formulation. Fluticasone propionate can be substituted by any of the drugs in Table 2, or a combination thereof. In addition, the omega 3 and omega 6 fatty acids listed in Table 3 and propellants listed in Table 4 can be used in the compositions and methods of the invention.

TABLE 1
Aerosol Formulations of Fluticasone Propionate (FP)

| | Example # | | | | |
|---|---|---|---|---|---|
| Ingredient (%) | 1 | 2 | 3 | 4 | 5 |
| FP | 0.0833 | 0.163 | 0.325 | 0.163 | 0.325 |
| Isopropyl linoleate | 0.0146 | 0.0146 | 0.0146 | 0.028 | 0.057 |
| HFA-134a | 99.902 | 99.82 | 99.66 | 99.809 | 99.618 |

Isopropyl Linoleate (0.365 g) was added directly to the open pressure vessel, which was then sealed. About 50% of the HFA 134a was added to the pressure vessel, which was stirred for 15 minutes to allow for solubilization to occur. Fluticasone propionate was added to a 0.5 L dispensing vessel situated at the top of the pressure vessel. The powder was then flushed into the formulation tank with the remaining HFA 134a. The tank was kept under constant stirring by an in-tank mixer set at 500 rpm. The suspension was then homogenized through an in-line high shear homogenizer set at ˜3200 rpm for 15 minutes, after which the contents of the transfer lines were flushed back into the formulation tank using nitrogen. After homogenization, the formulation tank was fitted to a double diaphragm pump filler via lines that transferred the formulation to the filler and then cycled back though the top of the tank. Valves were pre-crimped onto empty canisters and then filled under pressure with approximately 10.6 g of product. A total of 150 units each were filled from the beginning and end of the batch.

TABLE 2
Exemplary Drugs

Albuterol Sulphate (micronized)
Beclomethasone Dipropionate (micronized)
Mometasone Furoate (micronized)
Budesonide (micronized)
Triamcinolone Acetonide (micronized)
Ipratropium Bromide (micronized)
Formoterol Fumarate (micronized)
Salmeterol Xinofoate (micronized)
Cromolyn Sodium (micronized)

TABLE 3
Exemplary Omega 3 and Omega 6 Fatty Acids

Isopropyl Ester Omega -3 Linoleic acid
Isopropyl Ester Omega -3 Linolenic acid
Isopropyl Ester Eicosoapentaennoic Acid

TABLE 4
Exemplary Propellants

1,1,1,2 Tetrafluoroethane (HFA 134a)
1,1,1,2,3,3,3, Heptafluoropropane (HFA 227)

### Example 2

### Preparation and Isolation of Coated Particles

Method A: General Procedure for Use with all Surfactants, which can be Solubilized in a Solvent that will not Dissolve the Drug.

Isopropyl Linoleate (0.5 grams) is dissolved in 100 ml of methylene chloride. When solubility has been reached, 5.0 grams of solid fluticasone propionate is added and the mixture is rapidly stirred at RT for 30 minutes. At this time, the solid is filtered and washed with a minimum amount (10 ml) of chilled methylene chloride. The recovered Fluticasone Propionate is assayed for drug and Isopropyl Linoleate content. In addition, the melting point, differential scanning calorimetry (DSC), Porosity, surface area, X-ray diffraction (XRD), and scanning electron microscopy (SEM) are determined to characterize the coated particles. These particles are used in the preparation of HFA suspension formulations by classical techniques.

Method B: A Process for Making Surface Coated Particles that is Part of the Finished Product Manufacturing Process.

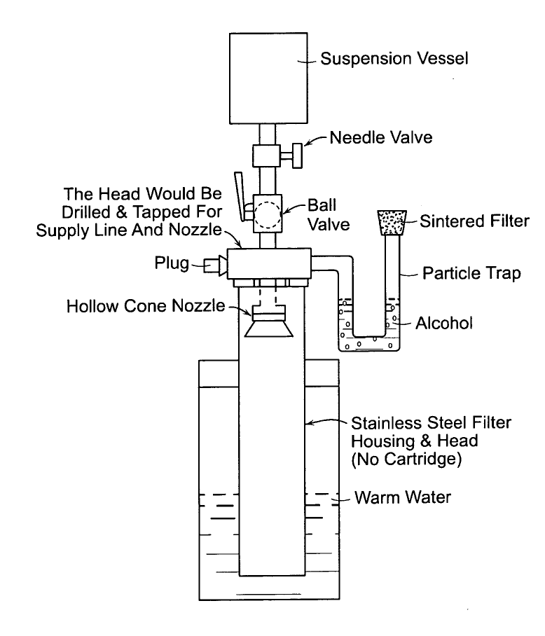

Isopropyl Linoleate (0.365 g) is added directly to an open pressure vessel that is then sealed. About 50% of the HFA 134a is added to the pressure vessel, which is stirred for 15 minutes to allow for solubilization to occur. Fluticasone propionate powder is added to a 0.5 L dispensing vessel situated at the top of the open pressure vessel. The powder is flushed into the formulation tank with the remaining HFA 134a. The tank is kept under constant stirring by an in-tank mixer set at 500 rpm. The suspension is then homogenized through an in-line high shear homogenizer set at ˜3200 rpm for 15 minutes after which the contents of the transfer lines are flushed back into the formulation tank using nitrogen. After homogenization, the formulation tank is fitted to a second vessel via a transfer line. This secondary vessel is fitted with an interior spray nozzle that causes the effluent to break up into micron size droplets. These droplets consist of propellant and coated fluticasone propionate. The secondary vessel is at 1 atmosphere, which causes the propellant to rapidly evaporate and the gas is vented. During the evaporation event, solid drug particles form and are deposited on the walls of the vessel. The walls can be heated to expedite the evaporation process, which may be useful when higher boiling point solvents are used in the compositions and processes. The deposited surface coated particles are collected and characterized by assay for drug and surfactant content, melting point, DSC, porosity, surface area, XRD, and SEM. These particles are used in the preparation of HFA suspension formulations by classical techniques. Alternatively, the secondary vessel that now comprises the deposited surface coated particles is attached to the final formulation/pressure vessel and the deposited powder is flushed directly, without isolation, into the formulation tank with the requisite HFA 134a. The final formulation tank is kept under constant stirring by an in-tank mixer set at 500 rpm. The vessels and tanks used in this example are shown in FIG. 1.

### INCORPORATION BY REFERENCE

The contents of all cited references (including literature references, patents, patent applications, and websites) that maybe cited throughout this application are hereby expressly incorporated by reference. The practice of the present invention will employ, unless otherwise indicated, conventional techniques of aerosol and drug particle formulation, which are well known in the art.

### EQUIVALENTS

The invention may be embodied in other specific forms without departing from the spirit or essential characteristics thereof. The foregoing embodiments are therefore to be considered in all respects illustrative rather than limiting of the invention described herein. Scope of the invention is thus indicated by the appended claims rather than by the foregoing description, and all changes that come within the meaning and range of equivalency of the claims are therefore intended to be embraced herein.

We claim:

1. A drug particle formulation comprising:
(a) a plurality of drug particles;
(b) a propellant substantially free of chlorofluorocarbons; and
(c) a soluble surfactant comprising an omega-3 and/or omega-6 fatty acid ester, wherein the omega-3 and/or omega-6 fatty acid ester prevents or reduces the amount of at least one of aggregation, agglomeration, caking, and precipitation.

2. The drug formulation according to claim 1, wherein the propellant comprises 1,1,1,2 tetrafluoroethane.

3. The drug formulation according to claim 1, wherein the propellant comprises 1,1,1,2,3,3,3 heptafluoropropane.

4. The drug formulation according to claim 1, wherein the propellant comprises 1,1,1,2 tetrafluoroethane and 1,1,1,2,3,3,3 heptafluoropropane.

5. The drug formulation according to claim 1, wherein the propellant comprises a non-chlorofluorocarbon chemical selected from the group consisting of a hydrocarbon, nitrogen, argon, nitrous oxide air, and carbon dioxide.

6. The drug formulation according to claim 5, wherein the hydrocarbon is selected from the group consisting of n-butane, isobutane, propane, pentane, isopentane, and isobutene.

7. The drug formulation according to claim 1, wherein the omega-3 and/or omega-6 fatty acid ester comprises a methyl ester.

8. The drug formulation according to claim 1, wherein the omega-3 and/or omega-6 fatty acid ester comprises an ethyl ester.

9. The drug formulation according to claim 1, wherein the omega-3 and/or omega-6 fatty acid ester comprises an isopropyl ester.

10. The drug formulation according to claim 9, wherein the omega-3 fatty acid ester comprises an isopropyl ester of omega-3 linoleic acid.

11. The drug formulation according to claim 1, wherein the omega-3 fatty acid ester comprises an isopropyl ester of alpha linoleic acid.

12. The drug formulation according to claim 1, wherein the omega-6 fatty acid ester comprises an isopropyl ester of linolenic acid.

13. The drug formulation according to claim 1, wherein the omega-3 fatty acid ester comprises an isopropyl ester of eicosapentaenoic acid.

14. The drug formulation according to claim 1, wherein the omega-3 fatty acid ester comprises an isopropyl ester of docosahexaenoic acid.

15. The drug formulation according to claim 1, wherein the omega-3 fatty acid ester is selected from the group consisting of an ester of linoleic, linolenic, eicosapentaenoic, and docoashexaenoic acid.

16. The drug formulation according to claim 1, wherein the omega-6 fatty acid ester comprises an isopropyl ester of gamma-linoleic acid.

17. The drug formulation according to claim 1, wherein the omega-3 and/or omega-6 fatty acid ester comprises about 0.001% to about 10% of the total weight of the drug formulation.

18. The drug formulation according to claim 1, wherein the omega-3 and/or omega-6 fatty acid ester comprises about 0.01% to about 1% of the total weight of the drug formulation.

19. The drug formulation according to claim 1, wherein the omega-3 and/or omega-6 fatty acid ester comprises about 0.01% to about 0.1% of the total weight of the drug formulation.

20. The drug formulation according to claim 1, wherein the omega-3 and/or omega-6 fatty acid ester comprises about 0.01% of the total weight of the drug formulation.

21. The drug formulation according to claim 1, wherein the drug formulation is a fluid.

22. The drug formulation according to claim 1, wherein the drug formulation is an aerosol suspension.

23. A delivery device comprising the drug formulation of claim 1.

24. The delivery device according to claim 23, wherein the delivery device comprises a container, a valve, and an actuator.

25. The delivery device according to claim 23, wherein the delivery device is a metered dose inhaler.

26. The delivery device according to claim 23, wherein the container comprises a material selected from the group consisting of coated and uncoated aluminium, steel, and glass.

27. A method for preparing a drug particle suspension comprising fine drug particles that are resistant to at least one of aggregation, agglomeration, caking, and precipitation, the method comprising the steps of:
(a) combining (i) an omega-3 and/or omega-6 fatty acid, or ester thereof, (ii) a propellant substantially free of chlorofluorocarbons, and (iii) a plurality of fine drug particles to form a particle suspension; and
(b) homogenizing the particle suspension, wherein the homogenized particle suspension is resistant to at least one of aggregation, agglomeration, caking, and precipitation.

28. The method according to claim 27, wherein the homogenizing step comprises high shear mixing.

29. The method according to claim 27, wherein the omega-3 and/or omega-6 fatty acid, or ester thereof, is combined with the propellant prior to being combined with the plurality of fine drug particles.

30. The method according to claim 27, wherein the method comprises the additional step of filtering or isolating the particle suspension.

31. The method according to claim 27, wherein the drug particles are pre-coated with an omega-3 and/or omega-6 fatty acid, or ester thereof, prior to being combined with the propellant.

32. The method according to claim 27, wherein the omega-3 and/or omega-6 fatty acid, or ester thereof, is dissolved in the propellant prior to being combined with the drug particles.

33. The method according to claim 27, wherein the propellant comprises 1,1,1,2 tetrafluoroethane.

34. The method according to claim 27, wherein the propellant comprises 1,1,1,2,3,3,3 heptafluoropropane.

35. The method according to claim 27, wherein the propellant comprises 1,1,1,2 tetrafluoroethane and 1,1,1,2,3,3,3 heptafluoropropane.

36. The method according to claim 27, wherein the omega-3 or omega-6 fatty acid ester comprises a methyl ester.

37. The method according to claim 27, wherein the omega-3 or omega-6 fatty acid ester comprises an ethyl ester.

38. The method according to claim 27, wherein the omega-3 or omega-6 fatty acid ester comprises an isopropyl ester.

39. The method according to claim 27, wherein the omega-3 or omega-6 fatty acid ester comprises a glycerol, sorbitol, or other alcohol ester.

40. The method according to claim 27, wherein the omega-3 fatty acid comprises alpha linoleic acid.

41. The method according to claim 27, wherein the omega-6 fatty acid comprises gamma linoleic acid.

42. The method according to claim 27, wherein the omega-3 fatty acid comprises linolenic acid.

43. The method according to claim 27, wherein the omega-3 fatty acid comprises eicosapentaenoic acid.

44. The method according to claim 27, wherein the omega-3 fatty acid comprises docosahexaenoic acid.

45. The method according to claim 27, wherein the particle suspension is a fluid.

46. The method according to claim 27, wherein the particle suspension is an aerosol suspension or solution.

47. The method according to claim 27, further comprising the step of isolating the surfactant coated drug particles.

48. The method according to claim 47, wherein the drug particles are isolated by filtration or precipitation.

49. A suspension of drug particles prepared according to the methods of claim 27.

50. A method for treating a respiratory, nasal, or systemic disorder, the method comprising the steps of:
(a) preparing a fine particle suspension according to the methods of claim 27; and
(b) administering the suspension to a mucous membrane in a patient.

51. A method for treating a respiratory or nasal disorder, the method comprising the steps of:
(a) preparing isolated drug particles according to the method of claim 47; and
(b) administering the isolated drug particles to a mucous membrane in a patient.

52. The method according to claim 50 or 51, wherein the mucous membrane is located in an oral, pulmonary, or nasal passage.

53. A method for preparing a coated drug particle, the method comprising the steps of:
(a) providing to a first vessel an omega-3 and/or omega-6 fatty acid;
(b) providing to the first vessel a propellant substantially free of chlorofluorocarbons;
(c) providing to the first vessel a plurality of fine drug particles to form a particle suspension;
(d) homogenizing the particle suspension;
(e) spraying the homogenized particle suspension onto a surface, thereby forming micron sized droplets comprising propellant and omega-3 and/or omega-6 fatty acid coated drug particles; and
(f) isolating the coated drug particles.

54. The method according to claim 53, wherein the surface comprises an interior wall of a second vessel.

55. The method according to claim 53, wherein the first vessel comprises a spray nozzle.

56. The method according to claim 54, wherein the second vessel comprises a spray nozzle.

57. The method according to claim 53, wherein the first vessel comprises a formulation tank, and the second vessel comprises a dispensing vessel in fluid communication with the formulation tank, wherein the formulation tank and the dispensing vessel are connected via a transfer line, wherein the fine drug particles are added to the dispensing vessel and are flushed into the formulation tank with a propellant via the transfer line.

58. The method according to claim 57, wherein the formulation tank is kept under constant stirring conditions.

59. The method according to claim 58, wherein the constant stirring conditions comprise about 500 rpm.

60. The method according to claim 57, further comprising the step of flushing the contents of the transfer line back into the formulation tank using nitrogen after the homogenizing step.

61. The method according to claim 54, wherein atmospheric pressure of the second vessel is about 0.001 to about 1 atmosphere.

62. The method according to claim 54, wherein atmospheric pressure of the second vessel is about 1 atmosphere.

63. The method according to claim 54, wherein the second vessel is kept at a temperature between about 10° C. to about 100° C., about 20° C. to about 40° C., or about 30° C. to about 40° C.

64. The method according to claim 53, wherein the isolating step comprises dessicating of the formulation.

65. The method according to claim 64, wherein during the dessicating step, the drug particles are deposited on the surface.

66. The method according to claim 54, wherein the isolating step comprises collecting the drug particles from the interior walls of the second vessel.

67. The method according to claim 54, further comprising the step of attaching the second vessel to a final formulation vessel.

68. The method according to claim 67, further comprising the step of flushing the coated particles from the second vessel into the final formulation vessel with a fluid.

69. The method according to claim 68, wherein the fluid is a propellant.

70. The method according to claim 68, wherein the fluid is a non-CFC propellant.

71. The method according to claim 68, wherein the fluid is a CFC propellant.

72. The method according to claim 68, wherein fluid is selected from the group consisting of HFA-134a and HFA-227, or a combination thereof.

73. The method according to claim 53, wherein the steps are performed in the order of (a)-(b)-(c)-(d)-(e)-(f).

74. The method according to claim 53, wherein the steps are performed in the order of (b)-(a)-(c)-(d)-(e)-(f).
